(19) United States
(12) Patent Application Publication
(10) Pub. No.: US 2024/0189003 A1
(43) Pub. Date: Jun. 13, 2024

(54) Construct for Bone Plate Modularity and Augmentation

(71) Applicant: Albert C. Lynch, Landenberg, PA (US)

(72) Inventor: Albert C. Lynch, Landenberg, PA (US)

(21) Appl. No.: 18/522,204

(22) Filed: Nov. 28, 2023

Related U.S. Application Data
(60) Provisional application No. 63/431,641, filed on Dec. 9, 2022.

(51) Int. Cl. A61B 17/80; A61B 17/86

(52) U.S. Cl. CPC A61B 17/8057; A61B 17/8042; A61B 17/86; A61B 2017/8655

(57) ABSTRACT

Methods and systems related to osteosynthesis are discussed herein. A construct for bone plate modularity and augmentation is disclosed. As used herein, the term “construct” refers to the functional combination of two or more implants, such as bone screws and bone plates. The construct includes a bone plate having a bone-facing surface and an opposite surface, at least one hole through the bone plate from the bone-facing surface to the opposite surface, and a primary threaded element. The primary threaded element extends through the hole, spans between the bone-facing surface and the opposite surface, and has a tip located on a side of the bone plate. The construct also includes a threaded interface separate from the bone plate, located on the side of the bone plate, and locking the primary threaded element in place in the construct.

### CROSS REFERENCE TO RELATED APPLICATIONS

This application claims the benefit of U.S. Provisional Patent Application No. 63/431,641, filed Dec. 9, 2022, which is incorporated by reference herein in its entirety for all purposes.

### BACKGROUND

The need to stabilize two or more segments of bone, in the field of surgical orthopedics, is frequently encountered. Broadly speaking, this may be performed using external or internal skeletal fixation. With reference to internal skeletal fixation, intra-medullary devices, which travel within the medullary canal of a bone, and bone plates, which affix to, or in close apposition to, the surface of the bone (the periosteum) via screws/bolts, are two of the most common implant technologies. As a bone plate sits on the surface of the bone, they are loaded eccentrically during weight bearing by the patient. This exposes the bone plate to bending, tensile, rotational and shear forces that they must absorb as the bone heals. These bone plates are commonly affixed to bone segments using bone screws.

### SUMMARY

Methods and systems related to osteosynthesis are discussed herein. In specific embodiments of the invention, a construct for bone plate modularity and augmentation is provided. As used herein, the term “construct” refers to the functional combination of two or more implants, such as bone screws and bone plates. In specific embodiments, a mechanism for mechanical coupling of bone plate implants is provided. In specific embodiments a threaded coupling entity is positioned on one surface of a bone plate, a screw is inserted from the opposing surface of the bone plate that spans the interposed bone plate, and the screw and threaded coupling entity compress the bone plate as the screw is tightened on the threads of the entity on the opposing surface. In specific embodiments, a construct is provided. The construct comprises a bone plate having a bone-facing surface and an opposite surface and at least one hole through the bone plate from the bone-facing surface to the opposite surface. The construct also includes a screw extending through the hole, spanning between the bone-facing surface and the opposite surface, and having a screw tip located on a side of the bone plate. The screw can extend from the bone-facing surface to the opposite surface or the opposite surface to the bone-facing surface in different embodiments. The construct can also comprise a threaded interface separate from the bone plate, located on the same side of the bone plate as the screw tip, and locking the screw in place in the resultant angle-stable construct.

Specific embodiments of the invention provide various benefits in the alternative or in combination. These benefits include the ability to increase the strength of a construct that otherwise could have been formed using a bone plate, the ability to modify a conventional bone plate and screw into an angle-stable locking bone plate construct, and the ability to customize the strength and/or footprint of an existing bone plate. The ability to customize the strength and footprint of a construct based on the real-time mechanical demands of a surgical procedure can reduce implant stock demands, cost, and procedural times, and can also result in better patient outcomes and faster healing times.

### BRIEF DESCRIPTION OF THE DRAWINGS

The accompanying drawings illustrate various embodiments of the systems, the methods, and various other aspects of the disclosure. A person with ordinary skills in the art will appreciate that the illustrated element boundaries (e.g., boxes, groups of boxes, or other shapes) in the figures represent one example of the boundaries. It may be that in some examples one element may be designed as multiple elements or that multiple elements may be designed as one element. In some examples, an element shown as an internal component of one element may be implemented as an external component in another and vice versa. Furthermore, elements may not be drawn to scale. Non-limiting and non-exhaustive descriptions are described with reference to the following drawings. The components in the figures are not necessarily to scale, emphasis instead being placed upon illustrating principles.

FIG. 1 illustrates locking and non-locking bone screws in accordance with the related art.

FIG. 2 illustrates a construct in accordance with specific embodiments of the invention disclosed herein.

FIG. 3 illustrates a first view and a second view of a construct in accordance with specific embodiments of the invention disclosed herein wherein a standard non-locking bone screw serves as the primary threaded element of the construct.

FIG. 4 illustrates various views of a standard bone plate and two bone screws that are in accordance with the related art.

FIG. 5 illustrates a view of a construct illustrating the density of bone screws relative to the length of the bone in accordance with the related art.

FIG. 6 illustrates various views of constructs being used with a non-anatomically reconstructed bone segment in accordance with the related art.

FIG. 7 illustrates various views of a two-piece supporting element, and components thereof, that are in accordance with specific embodiments of the inventions disclosed herein.

FIG. 8 illustrates various views of a two-piece supporting element attached to a bone plate at two points in accordance with specific embodiments of the inventions disclosed herein.

FIG. 9 illustrates various views of a construct, comprising a two-piece supporting element attached to a bone plate at two points, attached to a non-anatomically reconstructed bone segment in accordance with specific embodiments of the inventions disclosed herein.

FIG. 10 illustrates a construct, comprising a two piece-supporting element, a conventional bone plate, and a situational plate, being assembled in accordance with specific embodiments of the inventions disclosed herein.

FIG. 11 illustrates various views of a construct, comprising a two piece-supporting element, a conventional bone plate, and a situational plate, attached to a non-anatomically reconstructed bone segment in accordance with specific embodiments of the inventions disclosed herein.

FIG. 12 illustrates various views of a coupling element and primary threaded component in accordance with specific embodiments of the inventions disclosed herein.

FIG. 13 illustrates various views of a coupling element, in accordance with the coupling element of FIG. 12, being used to secure a two-piece supporting element, in accordance with the two-piece supporting element of FIG. 12 to a bone plate in accordance with specific embodiments of the inventions disclosed herein.

FIG. 14 illustrates various views of a primary threaded component being used to secure supporting elements to a bone plate where at least one of the supporting elements includes a threaded interface in accordance with specific embodiments of the inventions disclosed herein.

### DETAILED DESCRIPTION

Reference will now be made in detail to implementations and embodiments of various aspects and variations of systems and methods described herein. Although several exemplary variations of the systems and methods are described herein, other variations of the systems and methods may include aspects of the systems and methods described herein combined in any suitable manner having combinations of all or some of the aspects described.

Methods and systems related to osteosynthesis in accordance with the summary above are disclosed in detail herein. The methods and systems disclosed in this section are nonlimiting embodiments of the invention, are provided for explanatory purposes only, and should not be used to constrict the full scope of the invention. It is to be understood that the disclosed embodiments may or may not overlap with each other. Thus, part of one embodiment, or specific embodiments thereof, may or may not fall within the ambit of another, or specific embodiments thereof, and vice versa. Different embodiments from different aspects may be combined or practiced separately. Many different combinations and sub-combinations of the representative embodiments shown within the broad framework of this invention, that may be apparent to those skilled in the art but not explicitly shown or described, should not be construed as precluded.

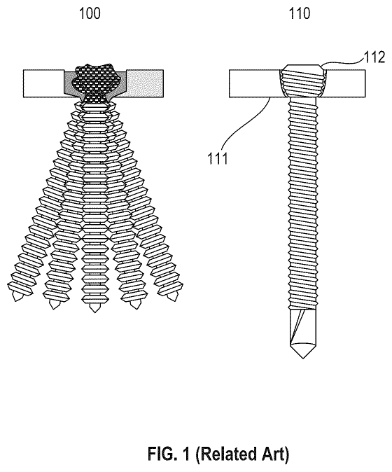

Bone plates generally require a minimum of two bone screws to be affixed to a bone segment. These bone screws may be non-locking, in which case their threads pull the bone to the bone surface of the bone plate, which achieves a mechanical interface with the bone via friction. These bone screws may alternatively be locking, in which case they have a coupling mechanism to the bone plate itself and results in a rigid bone-screw-to-bone-plate construct with a fixed angle, leading to a cantilever mechanical interface with the engaged bone. An example of a non-locking screw and a locking screw are provided in FIG. 1. Illustration 100 shows how non-locking screws can pass through the screw hole of a bone plate at different angles or can shift their angle relative to the surfaces of the bone plate after being inserted into the bone. Illustration 110 shows how a locking screw is only able to pass through the screw hole of the bone plate at a given angle and how a coupling mechanism, such as the threads in the bone plate 111 and the corresponding threads in the head of the screw 112, results in a rigid bone-screw-to-bone plate construct with a fixed angle.

In specific embodiments of the invention, the constructs disclosed herein can augment a conventional bone plate to include a locked, angle-stable, screw configuration. In specific embodiments of the invention, the constructs disclosed herein include bone plates with integrated threaded elements that act to create a locked screw configuration. The screws used in these sets of embodiments can be traditional bone screws, construct screws that fix portions of the construct to each other but do not penetrate the bone, or locking screws that lock both with a locking mechanism of their own and the threaded elements disclosed herein.

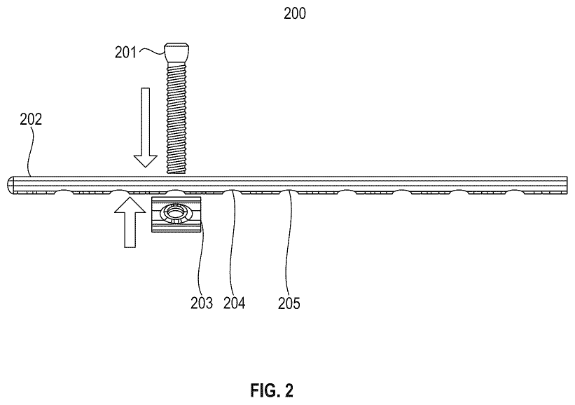

FIG. 2 illustrates a construct 200 in accordance with specific embodiments of the invention disclosed herein. Construct 200 includes a primary threaded component, which in this case is screw 201, which is introduced from one surface (e.g., a bone-facing surface or an opposite surface) of a bone plate 202. Construct 200 also includes a coupling element 203 which can include a threaded interface.

In specific embodiments, the primary threaded component will be inserted through a pre-existing hole in the bone plate. The pre-existing hole can be one of several pre-existing holes, such as hole 204 and hole 205 that are along the length of the bone plate for purposes of providing multiple possible locations for bone screws to be inserted through the bone plate. In alternative embodiments, the primary threaded component could alternatively span on the lateral side margins of the bone plate and the bone plate need not have pre-existing holes. The threads of screw 201 can span bone plate 202 to engage a threaded interface located on the opposite surface of bone plate 202 from where the primary threaded component was inserted. The threaded interface can include tapped threads that are designed to interface with the threads of screw 201.

The coupling element 203 can include a raised annulus which is shaped to interface with the set of holes in a bone plate at specific locations along the length of the bone plate. Alternatively, or in combination, coupling element 203 could include baffles or clips that are formed to connect with the sides of the bone plate to keep the coupling element 203 in place prior to the insertion of the primary threaded component. These features can serve to minimize slack between coupling element 203 and bone plate 202.

The primary threaded element and threaded interface can lead to increased structural stability of the resulting construct. The primary threaded component can include a tip that is inserted into the hole of the bone plate such that it will thereby be located on the same side of the bone plate as the threaded interface. Screw 201 is unable to completely pass-through bone plate 202 to its opposing surface where the threaded interface is positioned. Therefore, as the threads of screw 201 continue to engage the receptive tapped threads of the threaded interface, up to the point where screw 201 is mechanically unable to pass through bone plate 202 any further, the components are lagged (i.e., compressed) together, thus mechanically coupling them via resultant frictional forces.

In the illustrated example, the threaded interface is part of a separate component such as the support elements or coupling elements disclosed below. As such, in the illustrated case all three elements (screw 201, bone plate 202, and the coupling element 203) are lagged together. However, in specific embodiments of the invention, the threaded interface will be a part of the bone plate itself and a separate element is not required.

The embodiments described with reference to FIG. 2, and others like it can add locking functionality to existing bone plate constructs or increase the strength of the locking mechanism of those that already include locking functionality. For example, bone plate 202 does not have threading in the hole through the bone plate that is meant to fit that of screw 201 and is a conventional bone plate. However, the threading provided by the separate component, which can be a simple component with threading and a profile to mate with the bone plate, modifies the bone plate to have locking functionality. As another example, if a traditional, non-locking, conventional bone screw is used as screw 201, the illustrated coupling mechanism will still result in an angle stable, locked, construct with the three components locked together in the construct. As another example, if a locking screw were to be used as screw 201, such as one that press fits into bone plate 202 via a morse taper screw head (as seen), then this coupling mechanism adds an additional locking mechanism, complementary to its existing locking technology, to thereby improve its frictional press fit.

In specific embodiments of the invention, the primary threaded component can be inserted from the bone-facing surface and the threaded interface can be on the side of the opposite surface. In the context of bone surgery, the opposite side to the bone-facing surface can be referred to as the surgeon-facing surface. In specific embodiments of the invention, the primary threaded component can be inserted from the surgeon-facing surface and the threaded interface can be on the side of the bone-facing surface. In specific embodiments, the primary threaded component is inserted before the bone plate is placed in contact with the bone such that either surface of the bone plate is exposed and accessible. The construct can be formed prior to contact with the bone as a stored generic bone plate is being customized for a specific situation using the approaches disclosed herein. In alternative embodiments, the constructs can be formed while the bone plate is in contact with the bone as a bone screw is used as the primary threaded component and it engages with the threaded interface as the bone screw is being inserted into the bone.

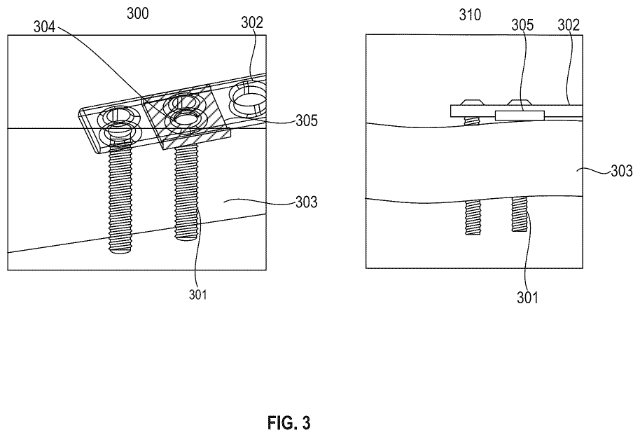

FIG. 3 illustrates a first view 300 and a second view 310 of a construct in accordance with specific embodiments of the invention disclosed herein wherein a standard non-locking bone screw serves as the primary threaded element of the construct. In first view 300 bone plate 302, primary threaded element 301, and bone 303 are transparent to reveal a threaded interface 304 on a coupling element 305. In second view 310 bone plate 302 and primary threaded element 301 are shown with the primary threaded element 301 being a bone screw that extends through bone 303. In the illustrated case, bone plate 302 and primary threaded element 301 are non-locking elements. As such, without the threaded interface 304 of coupling element 305 bone plate 302 and primary threaded element 301 would otherwise form a bone-screw friction construct. However, in the illustrated case, the construct is a fixed angle construct as the primary threaded element 301 is lagged with bone plate 302 and is locked in place across the entire width of the hole through bone plate 302. The illustrated construct can be used in patients with osteoporotic bone, to provide conversion of a non-locking construct (e.g., a conventional non-locking bone plate and bone screw) to a locking construct. Osteoporotic bones are thin; therefore, non-locking constructs that depend on screw-thread-to-bone engagement and frictional forces are less optimal. The illustrated construct may also be used when operating near a joint, where use of a locking screw trajectory may be beneficial to ensure that the screw does not go into the joint.

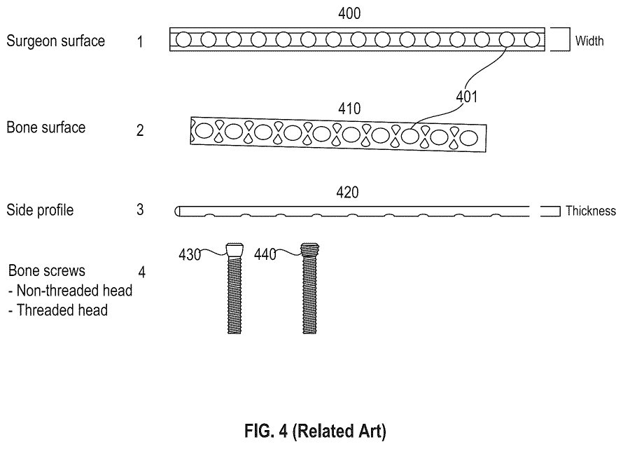

FIG. 4 provides various views of a standard bone plate and two bone screws that are in accordance with the related art. As used in this disclosure, the term standard bone plate will refer to a surgical implant which has the shape of an elongated rectangle with holes spaced along its length. It may be made from different materials, such as titanium or stainless steel. It is affixed to the surface of a bone. It has two surfaces, one that faces the bone and one that faces the surgeon. The side of a bone plate that faces the surgeon can be referred to as the surgeon-facing side. View 400 illustrates a surgeon-facing side of a standard bone plate. The side of a bone plate that faces the bone can be referred to as the bone-facing side. View 410 illustrates a bone-facing side of the standard bone plate in view 400. View 420 illustrates a side view of the standard bone plate in view 400. As used herein the term bone screw refers to a screw having a head and a shank, either of which may have thread present. Bone screw 430 is a non-locking screw and has threads on the shank of the screw. Bone screw 440 is a locking screw and has threads on both the shank and the head of the screw. The end of the shank can be referred to as the tip of the bone screw. The shank passes through the holes in the bone plate to engage the bone. Holes 401 scan be seen in view 400 and view 410. The head of the bone screw is larger than the hole in the bone plate and does not pass through. The holes in the bone plate may be threaded or non-threaded. The surfaces of the bone plate may have surface contours, such as undercut leaflets on the bone surface of the bone plate as shown in view 410.

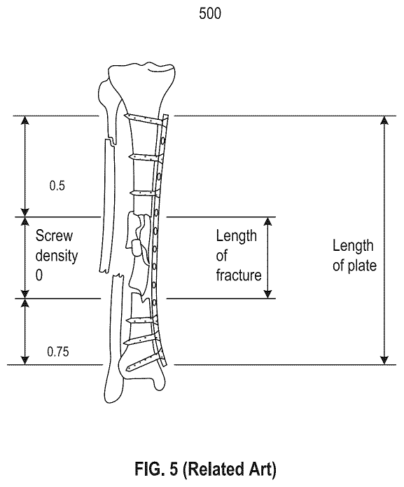

Bone plates are most often used to stabilize two or more segments of bone during osteosynthesis. For example, two segments of bones that have been severed at a demarcation due to a fracture or osteotomy. One end of the bone plate is affixed to one bone segment, while the other end of the bone plate is affixed to another bone segment. The bone plate is exposed to compressive, tensile, rotational and shear forces as the patient loads the bone segments. The mechanical strength of the bone plate under these loading conditions is directly proportionate to the width and thickness of the bone plates available material volume raised to the third power. Regions of the bar stock, from which the bone plate is manufactured, that have holes present for passage of a bone screw or other implant, have significantly reduced area moment of inertia (AMI) due to the reduced cross-sectional material volume. Therefore, regions with holes in the bone plate that are not required for passage of a bone screw to engage bone, and are exposed to forces during loading, weaken the bone plate implant and make it at high risk for failure unless additional implants are placed. FIG. 5 is illustrative in this regard as the illustrated holes in the center of view 500 span the area of fracture even though the density of screws in this area is zero.

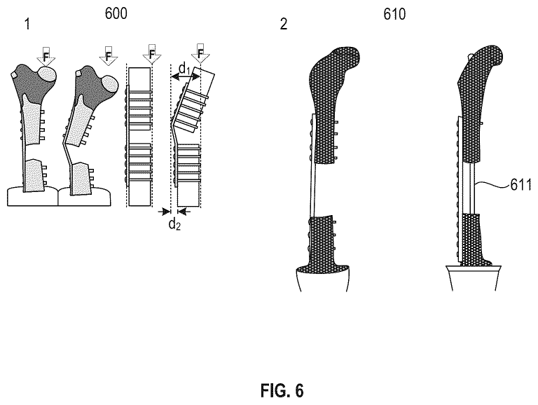

When a bone is fractured and the bone segments cannot be anatomically reconstructed, then all forces through the bone segments during loading are transmitted to the construct that is stabilizing those bone segments. Many times, this situation also leads to the mechanically inopportune situation mentioned in the prior paragraph, wherein the bone plate has open screw holes located over an area of fracture, but in this situation the screw holes are located over a non-anatomically reconstructed region as shown in view 600 of FIG. 6. As illustrated in that view, this can lead to catastrophic failures of the implant as the impact of load bearing forces on the opposite side of the bone are levered by the width of the bone screws contacting the bone. In situations where the surgeon has an open screw hole over the non-anatomically reconstructed fracture site, it may be addressed in several ways. First, the surgeon may elect to use a larger bone plate that affords sufficient strength, even at the weak open screw hole locations. This leads to an excessively stiff implant at other sites of the bone that may cause a stress riser from oversized bone screw placement, predisposing to subsequent implant related fracture, and stress shielding, where the bone becomes poorly mineralized and weak beneath the bone plate because the bone has not had appropriate loads transmitted to it. Alternatively, the surgeon may place an additional implant, such as a second bone plate or intra-medullary device 611 as shown in view 610. However, this requires additional surgical approaches, which can impede healing and lead to patient discomfort, protract anesthesia/surgery time and increased cost. As another option, the surgeon may elect a specific type of bone plate with a solid central region of bar stock, without holes present, called a lengthening plate. This demands a high implant reservoir to ensure that it has the right amount of holes, length and solid bar stock spanning sections.

In specific embodiments of the invention a support element can be added to a bone plate via any of the primary threaded elements and threaded interfaces mentioned above. For example, the support element can be connected to the bone plate via a primary threaded element in the form of a screw that passes through the bone plate but does not extend beyond the support element, or via a bone screw that extends into a bone of the patient. The support element can comprise a single piece located on either side of the bone plate or two pieces with one on each side of the bone plate. The support element can cover a portion of the bone plate with holes that extend over a fracture area or region of non-anatomical reconstruction. The support element, and each piece of the support element, can include a profile that mirrors that of the bone plate to which it will support. For example, it can extend from one edge of the bone plate to the other. In the alternative or in combination, it can include protrusions shaped to engage with the holes of the bone plate or other surface contours of the bone plate such as undercut leaflets. The support elements, and each piece of the support element, can include through holes. The through holes can align with holes on the bone plate and receive a primary threaded element such as those mentioned above. The threaded interface can be part of the support elements, and each piece of the support element, or they can be part of a separate element used to connect the support element with the bone plate. The through holes can be configured to be on either side of a support element which spans a fracture area or region of non-anatomical reconstruction.

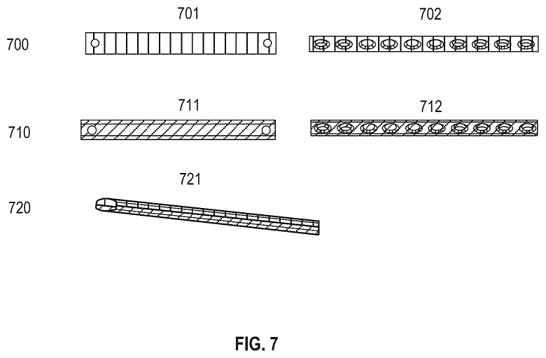

FIG. 7 below provides several views of a two-piece supporting element that is in accordance with specific embodiments of the inventions disclosed herein. A first piece of the supporting element 700 is shown in view 701 and view 702. A second piece of the supporting element 710 is shown in view 711 and 712. The two-piece supporting element 720 is shown in view 721 with piece 700 joined together with piece 710. The supporting element can adopt any desired profile. However, in the illustrated case the supporting element has an elongated rectangular profile and reflects the bone plate to which it will be applied. The supporting element in this situation can be referred to as a coupling plate due to its profile and the existence of more than one hole. As a result, the supporting element in this case can be used either to strengthen a portion of a single bone plate, to join to separate bone plates together into a single construct, or to join a bone plate with a coupling element such as a customized joint attachment, or situational plate, as will be described further below. In alternative embodiments, the supporting element can have a single hole and extend in one or two directions from that hole while still increasing the strength of the portion of the bone plate to which it is attached.

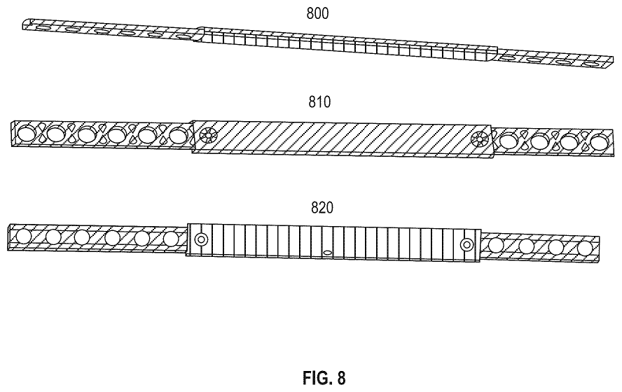

Returning to the illustrated example in FIG. 7, the profile of the support element can have an exterior surface, opposite the bone plate, and an interior surface, which faces the bone plate. The exterior surface of piece 700 is shown in view 701. The exterior surface of piece 710 is shown in view 711. The exterior surface of piece 700 is smooth and is intended to face a surgeon-facing side of an implant. The exterior surface of piece 710 includes a groove and is intended to face a bone-facing side of an implant with the groove being at least partially conformal to the bone of the patient. The interior surface can have a detailed footprint reflecting that of the bone plate to which it will be applied, thus increasing contact and frictional coupling. The interior surface of piece 700 is shown in view 702 and includes raises annular elements that are configured to interface with the holes in one or more bone plates to which the piece will be attached. The interior surface of piece 710 is shown in view 712 and includes raises annular elements that are configured to interface with the holes in one or more bone plates to which the piece will be attached. Alternatively, or in combination, the pieces of a supporting element in accordance with this disclosure could include baffles or clips that are formed to connect with the sides of the bone plate to keep the supporting element in place prior to the insertion of the primary threaded component. Alternatively, or in combination, the support element or pieces thereof could span the width of the bone plate and grip the bone plate on a first edge of the bone plate and a second edge of the bone plate as shown below in FIG. 8. The support element may be applied to the surface of the bone plate on the bone-facing side 711, the surgeon-facing side 701, or both such that the support piece 720 sandwiches one or more bone plates.

Any combination of coupling plates, coupling mechanisms and directionality of the applied surfaces can be utilized to enhance the strength of a bone plate. By mechanically coupling the applicable components of specific embodiments of the inventions disclosed herein to regions of a bone plate that are perceived to be of insufficient mechanical strength to withstand the load and forces to which they will be exposed to upon weight bearing by the patient, the surgeon is able to increase the strength/AMI of the construct in a targeted manner and potentially negate the necessity of additional secondary implants.

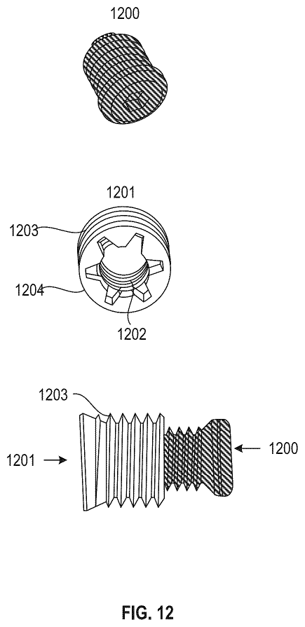

FIG. 8 illustrates an example of a two-piece supporting element attached to a bone plate at two points. Views 800, 810 and 820 show how two-pieces of the supporting element are attached using attachment elements that take the form of those described below with reference to FIG. 12. As described below, the two-pieces of the supporting element could each included a threaded interface such that they formed a locking configuration with a screw or other threaded attachment means that was used to connect the two pieces of the supporting element together on either side of the bone plate.

The span of the supporting element in FIG. 8 between the two points at which the supporting element pieces are attached to each other and the bone plate could mirror the span of a fracture area on the bone to which the bone plate is being applied. As such, the supporting elements enhance the strength of the bone plate at the area in which the holes of the bone plate would otherwise present an area of mechanical weakness in a region that required high mechanical strength.

Any combination of coupling plates, coupling mechanisms and directionality of the applied surfaces can be utilized to enhance the strength of a bone plate. By mechanically coupling the applicable components of specific embodiments of the inventions disclosed herein to regions of a bone plate that are perceived to be of insufficient mechanical strength to withstand the load and forces to which it will be exposed to upon weight bearing by the patient, the surgeon is able increase the strength/AMI of the construct in a targeted manner.

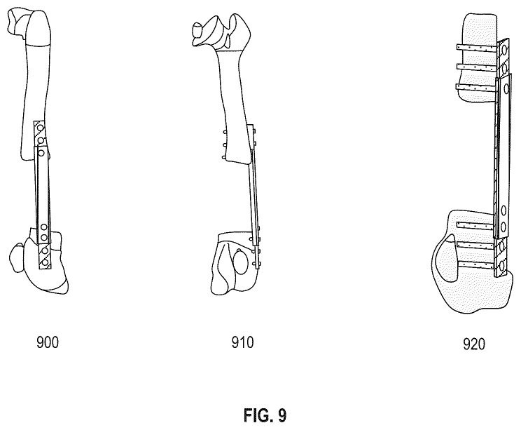

FIG. 9 provides an example of the usage of a bone plate with a supporting element in accordance with specific embodiments of the invention disclosed herein being used to span a non-anatomically reconstructed region of a patient's bone. View 900 and view 910 of FIG. 9 provide orthogonal image renderings of a simulated femur fracture. The non-anatomic reconstructed area of bone is removed to represent the mechanical environment wherein all forces are transmitted through the implant. An approach in accordance with specific embodiments of the inventions disclosed herein has been applied to attach a supporting element comprising two pieces to the central region of the bone plate where increased mechanical strength of the construct is desired. The primary threaded elements mechanically linking the two pieces of the supporting element to the bone plate are seen engaging the proximal (top) and distal (bottom) bone segments. View 920 includes a transparent rendering of the bone to demonstrate the screw trajectory through the bone. As illustrated, the supporting element pieces are connected to the bone plate via both bone screws and a single screw that does not extend into the bone.

When the demarcation (such as a fracture or osteotomy) between two or more bone segments is located near the end of the bone, the straight elongated rectangular geometry of a conventional bone plate no longer fits the profile of the bone segment that needs to be engaged by bone screws. This can lead to poor alignment, implant failure and patient morbidity. Situational plates that are designed to fit specific anatomic regions exist, but conventional situational plates demand an extensive implant reservoir to ensure the correct sidedness and length. Additionally, these situational plates are similarly at risk of low mechanical strength at key locations due to “open holes” over a region of non-anatomic reconstruction as are standard rectangular bone plates.

In specific embodiments of the inventions disclosed herein, a situational plate can be connected to a bone plate via the primary threaded components and threaded interfaces mentioned above. The situational plate can have one of a t-shaped profile, a clover leaf shaped profile, a patient-specific profile or other anatomically advantageous profile for a given application. Patient-specific and anatomically advantageous profiles can be set based on the characteristics of the bone proximate to a joint where the bone plate will be connected. The situational plate can be a fixed length which is shorter than those found in a typical reservoir of conventional situational plates. In these embodiments, the construct effectively alters an interface footprint of the bone plate and increases the amount of customization available to surgeons with a given implant reservoir.

The situational plate can be fixed in the construct in various ways. In specific embodiments of the invention, the situational plate can be attached to a bone plate by virtue of both plates being attached to a supporting element in accordance with the approaches disclosed above (e.g., those of FIGS. 7 and 8). The situational plate and the supporting element can be connected via a primary threaded component and threaded interface using any of the approaches disclosed above. In specific embodiments of the invention, the situational plate can be attached to a bone plate by virtue of the threaded interface being a part of the situational plate. The threaded interface can be part of the supporting element, part of the situational plate, or part of a separate coupling element.

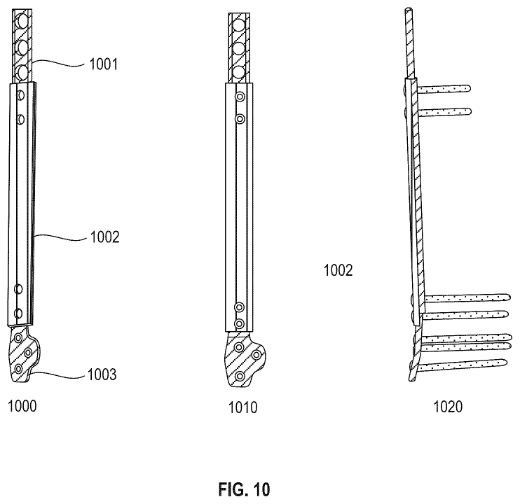

FIG. 10 provides an example of several views 1000, 1010, and 1020 of a construct including a conventional rectangular bone plate 1001, a supporting element 1002, and a situational plate 1003 where supporting element 1002 connects bone plate 1001 and situational plate 1003. In this example, situational plate 1003 is connected to supporting element using a primary threaded component in the form of bone screws. However, in alternative embodiments, the supporting element can be connected to the situational plate using threaded components that do not extend into the bone or a combination of threaded components that do not extend into the bone and bone screws. Any combination of coupling plates, coupling mechanisms and the directionality of the applied surfaces are applicable to examples in accordance with the construct in FIG. 10 as explained above. By mechanically coupling a supporting element in this manner to the end of a bone plate, a surgeon is able to customize the profile footprint, and therefore optimize screw engagement of the associated bone segment without access to an unwieldy large library of previously formed constructs. The situational plates can have different lengths and different contours for interfacing with different bone surfaces or joints, or different aspects thereof, in order to increase the flexibility afforded to surgeons by a library of implements for the attachment of a given bone plate to a given patient. If more than one coupling plate is used, then they may continue beyond the end of the interposed bone plate, to minimize the stress riser at that transition point (the location where the interposed bone plate ends).

In view 1000, the situational plate is a custom coupling plate with a t-shaped shaped profile. The situational plate could be a custom coupling plate with different profiles such as a clover leaf, flat, or t-shaped profile. The custom coupling plate could also include a solid lengthening component. Different custom extensions with varying profiles such as different situational plates with configurations such as t-shapes and other profiles can be easily switched out and connected to a supporting element using the approaches disclosed herein. In view 1020, a side profile of the construct is provided with bone screws included. As illustrated, the bone screws both attach the supporting element to the conventional bone plate and the situational bone plate and attach the entire construct to the bone. The bone screws thereby both fix the construct to the bone and fix the portions of the construct together.

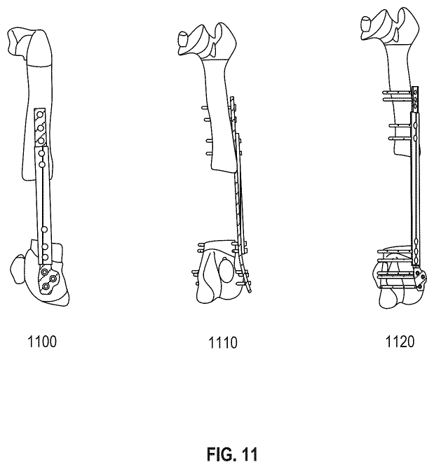

FIG. 11 provides an example of one of the constructs in FIG. 10 being applied to span a non-anatomically reconstructed portion of a patient's bone. The left and center images are orthogonal image renderings of a simulated femur fracture. The non-anatomic reconstructed area of the bone is removed to represent the mechanical environment wherein all forces are transmitted through the implant. Specific embodiments of the inventions disclosed herein have been utilized to attach a situational plate with a special screw configuration to the distal segment of the femur, maximizing the screw engagement of this bone segment. A second piece of the secure element, to make the secure element two sided, is utilized to further increase the strength of the construct over the fracture site, as well as to mitigate the stress riser potential at the transition point where the bone plate ends.

In specific embodiments of the invention, the threaded interfaces mentioned above can be part of a separate coupling element that is used to attach one portion of the construct to the bone plate by interfacing with the primary threaded component. In specific embodiments of the invention, the construct can include a second threaded interface that is used to fix the same primary threaded component. The coupling element can include the second threaded interface. For example, the threaded interface can be on an inner surface of the coupling element and interface with the primary threaded component while the second threaded interface is on the outer surface of the coupling element. FIG. 12 below provides an illustration of such a coupling element in the form of secondary screw 1201. The secondary screw 1201 includes a threaded interface 1202 on an inside of secondary screw 1201 that is configured to interface with threads on a primary threaded component such as primary screw 1200. The secondary screw 1201 also includes a second threaded interface 1203 on an outside of secondary screw 1201 that is configured to interface with threads on a bone plate or supporting element such as those described above with reference to FIG. 7. While the example of FIG. 12 uses a primary screw 1200 that does not extend past the illustrated coupling element, the same coupling element of FIG. 12 (i.e., the secondary screw 1201) could be used with a bone screw that is designed to extend past the coupling element (e.g., past the non-threaded head 1204 of secondary screw 1201).

Specific embodiments of the invention may include one or more coupling mechanisms. As previously disclosed, at least one primary threaded component will engage a threaded interface located on an opposing surface of the bone plate. If this singular coupling mechanism is chosen, then the coupling threads may be present in a static component on the opposite surface of the bone plate. As discussed above, there may be one single coupling mechanism converting a conventional non-locking screw to a fixed angle locking construct. Also, as discussed above, there may be numerous components engaged via the single coupling mechanisms, with various arrangements. Regardless, more than one coupling mechanism can be used to connect the components. For example, a dual coupling mechanism may also be used. For example, in FIG. 12 a coupling element in the form of a secondary screw 1201 can be placed first that is capable of mechanically coupling all involved components, except for the primary screw. The non-threaded head 1204 of the secondary screw 1201 can mechanically anchor at one side surface of a bone plate, while the external threads 1203 of the secondary screw 1201 engage tapped threads of a component anchored to the opposing surface of the bone plate. The primary screw 1200 can subsequently be placed, engaging the internal threads 1202 of the secondary screw 1201. If the thread “handedness” of the primary and secondary screw are opposite, then placing them from opposing surfaces of the bone plate will result in a second mechanical coupling that enhances the coupling achieved by the first (i.e. as the primary screw is tightened, it also tightens the secondary screw further, maximizing the resultant frictional coupling).

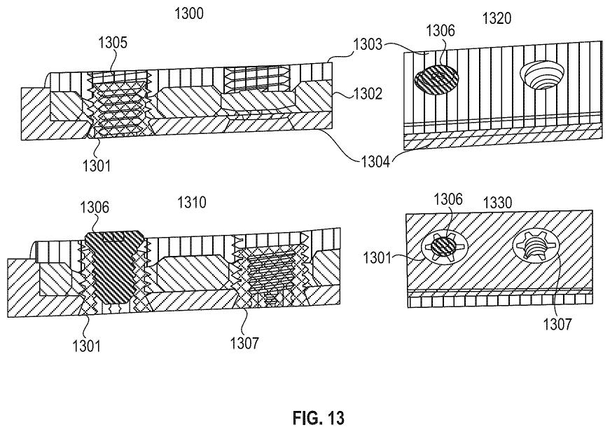

FIG. 13 provides a series of images of a coupling element in accordance with FIG. 12 attaching two pieces of a supporting element to bone plate. The direction of component placement is maintained in this diagram but can be interchanged in the direction that the primary and secondary screws are placed. The first view 1300 is a sectioned view of the coupling element in the form of a secondary screw 1301 placed through the bone plate 1302, first piece of the supporting element 1303, and second piece of the supporting element 1304. Secondary screw 1301 engages with a threaded interface 1305 in first piece of the supporting element 1303. Secondary screw 1301 is placed from the bone surface side of bone plate 1302 and engages thread in the threaded interface 1305 in the first piece of the supporting element 1303 on the surgeon surface side of the bone plate 1302. There is no thread present in the head of the secondary screw 1301, leading to frictional coupling. In view 1310, there is now one primary screw 1306 placed and two secondary screws—secondary screw 1031 and secondary screw 1307. Primary screw 1306 is being placed from the surgeon surface side of the bone plate 1302 and engages the internal thread of the secondary screw 1301 that is anchored on the bone surface side of the bone plate 1302. There is no thread present in the head of the primary screw, leading to frictional coupling. Furthermore, since the thread direction/handedness of the primary screw 1306 and secondary screw 1301 are in opposite directions, they are therefore complementary. As the primary screw 1306 tightens via engaging the internal threads of the secondary screw 1301, this exerts torque that further tightens the coupling of the external thread of secondary screw 1301. View 1320 and view 1330 show opposite sides of the construct in view 1310 with primary screw 1306 visible from both sides.

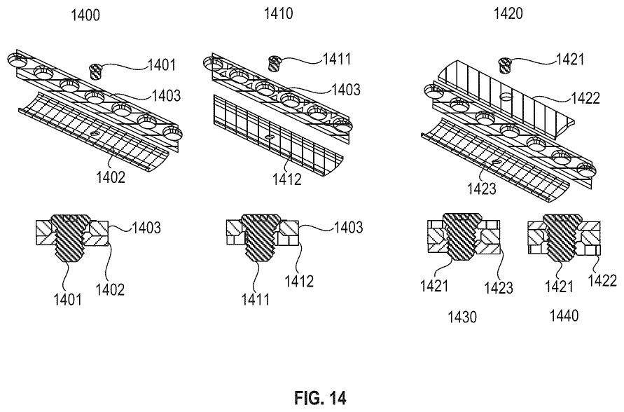

FIG. 14 illustrates various views of a primary threaded component being used to secure supporting elements to a bone plate where at least one of supporting elements includes a threaded interface in accordance with specific embodiments of the inventions disclosed herein. View 1400, view 1410, and 1420 all exhibit a single coupling mechanism and primary lag screw with interposed bone plate. View 1420 includes an additional coupling plate. As seen in view 1400 a primary screw 1401 is inserted through a bone plate 1403 and interfaces with a threaded interface on a supporting element 1402. The primary screw in this case is being placed from the surgeon facing surface of the bone plate. As seen in view 1410 a primary screw 1411 is inserted through a bone plate 1403 and interfaces with a threaded interface on a supporting element 1412. The primary screw in this case is being placed from the bone facing surface of the bone plate. As seen in view 1420, a primary screw 1421 is being placed through a bone plate 1403 and also extends at least partially through a first supporting element 1422 and a second supporting element 1423. The first supporting element 1422 and second supporting element 1423 can be two pieces of a two-piece supporting element. The primary screw in this case is being placed from a surgeon facing surface of the bone plate. The singular coupling mechanism may couple one or more components interfaced between the core coupling mechanism. For example, primary screw 1401 engages with a single threaded interface. As seen in view 1430 and view 1440, when the primary screw 1421 is placed through two different supporting elements either one or both of the supporting elements can include a threaded interface to interact with primary screw 1421. When more than one coupling plate is utilized such as in view 1420, the holes in the bone plate through which the primary screw travels can be glide holes, which do not engage the primary screw shank threads.

While the specification has been described in detail with respect to specific embodiments of the invention, it will be appreciated that those skilled in the art, upon attaining an understanding of the foregoing, may readily conceive of alterations to, variations of, and equivalents to these embodiments. For example, while the term supporting element was used to described components that were attached to the bone plate, the elements do not need to provide additional support to the bone plate and can simply be utilized to connect the bone plate to various different situational plates to allow for bone plates of various sizes to be accessorized for different applications. These and other modifications and variations to the present invention may be practiced by those skilled in the art, without departing from the scope of the present invention, which is more particularly set forth in the appended claims.

What is claimed is:

1. A construct comprising:
a bone plate having a bone-facing surface and an opposite surface;
at least one hole through the bone plate from the bone-facing surface to the opposite surface;
a primary threaded element: (i) extending through the hole; (ii) spanning between the bone-facing surface and the opposite surface; and (iii) having a tip located on a side of the bone plate; and
a threaded interface separates from the bone plate, located on the side of the bone plate, and locking the primary threaded element in place in the construct.

2. The construct of claim 1, wherein the hole through the bone plate does not include threading for the primary threaded element.

3. The construct of claim 1, wherein the primary threaded element is a bone screw.

4. The construct of claim 1, wherein the primary threaded element is a locking screw.

5. The construct of claim 1, wherein the bone-facing surface is on the side of the bone plate.

6. The construct of claim 1, wherein the opposite surface is on the side of the bone plate.

7. The construct of claim 1, further comprising:
a support element connected to the bone plate via the primary threaded element and the threaded interface.

8. The construct of claim 7, wherein:
the threaded interface is part of the support element.

9. The construct of claim 7, further comprising:
a coupling element;
wherein the threaded interface is part of the coupling element.

10. The construct of claim 7, wherein:
the support element spans a width of the bone plate and grips the bone plate on a first edge of the bone plate and a second edge of the bone plate.

11. The construct of claim 7, further comprising:
a set of holes in the bone plate;
wherein the support element covers the set of holes.

12. The construct of claim 7, wherein the support element comprises:
a first piece on the side of the bone plate; and
and a second piece on an opposite side of the bone plate.

13. The construct of claim 12, wherein:
the threaded interface is part of the first piece.

14. The construct of claim 7, wherein:
the support element comprises a first piece on the side of the bone plate and a second piece on an opposite side of the bone plate.

15. The construct of claim 7, further comprising:
a situational plate attached to the support element;
wherein the situational plate is attached to the bone plate via the support element.

16. The construct of claim 15, wherein:
the situational plate has one of a t-shaped profile, a clover leaf shaped profile, and another patient-specific profile.

17. The construct of claim 7, further comprising:
a situational plate attached to the support element;
wherein the situational plate is attached to the bone plate via the support element.

18. The construct of claim 1, further comprising:
a situational plate;
wherein the threaded interface is part of the situational plate.

19. The construct of claim 1, further comprising:
a coupling element;
wherein the threaded interface is part of the coupling element.

20. The construct of claim 19, further comprising:
a second threaded interface;
wherein the second threaded interface is part of the coupling element, the threaded interface is on an inner surface of the coupling element, the second threaded interface is on an outer surface of the coupling element.

21. The construct of claim 1, wherein:
without the threaded interface the bone plate and screw would otherwise form a bone-screw friction construct; and
the construct is a fixed angle construct.

22. The construct of claim 1, wherein:
the construct increases a strength of the bone plate over a fracture area.

23. The construct of claim 1, wherein:
the construct alters an interface footprint of the bone plate.

24. A construct comprising:
a bone plate having a bone-facing surface and an opposite surface;
at least one hole through the bone plate from the bone-facing surface to the opposite surface;
a primary threaded element: (i) extending through the hole; (ii) spanning between the bone-facing surface and the opposite surface; and (iii) having a tip located on a side of the bone plate;
a support element connected to the bone plate via the primary threaded element; and
a set of holes in the bone plate;
wherein the support element covers the set of holes.

25. The construct of claim 24, wherein:
the support element spans a width of the bone plate and grips the bone plate on a first edge of the bone plate and a second edge of the bone plate.

26. The construct of claim 24, further comprising:
a situational plate attached to the support element;
wherein the situational plate is attached to the bone plate via the support element.

27. A construct comprising:
a bone plate having a bone-facing surface and an opposite surface;
at least one hole through the bone plate from the bone-facing surface to the opposite surface;
a primary threaded element: (i) extending through the hole; (ii) spanning between the bone-facing surface and the opposite surface; and (iii) having a tip located on a side of the bone plate;
a support element connected to the bone plate via the primary threaded element; and
a situational plate attached to the support element;
wherein the situational plate is attached to the bone plate via the support element.
